(19) United States
(12) Patent Application Publication
(10) Pub. No.: US 2026/0171244 A1
(43) Pub. Date: Jun. 18, 2026

(54) AI-ASSISTED STROKE INTERPRETATIONS SYSTEM

(71) Applicant: Ever Fortune.AI CO., Ltd., Taichung City (TW)

(72) Inventor: TI-HAO WANG, Taichung City (TW)

(73) Assignee: Ever Fortune.AI CO., Ltd., Taichung City (TW)

(21) Appl. No.: 19/020,567

(22) Filed: Jan. 14, 2025

Related U.S. Application Data
(60) Provisional application No. 63/735,445, filed on Dec. 18, 2024.

(51) Int. Cl. G16H 50/20; A61B 6/00; A61B 6/46; A61B 6/50; G06T 7/00; G06V 10/764; G06V 10/774; G06V 10/778

(52) U.S. Cl. CPC G16H 50/20; A61B 6/467; A61B 6/501; A61B 6/5247; G06T 7/0016; G06V 10/764; G06V 10/774; G06V 10/7788; G06T 2200/24; G06T 2207/10081; G06T 2207/10088; G06T 2207/20081; G06T 2207/20092; G06T 2207/30016; G06T 2207/30096; G06V 2201/031

(57) ABSTRACT

A stroke AI-assisted interpretation system, configured to operate on a terminal device that includes a user interface, further comprising an image acquisition module for receiving medical imaging data from a patient's computed tomography (CT) and/or magnetic resonance imaging (MRI); an image preprocessing module for preprocessing the medical imaging data; an artificial intelligence engine, trained on a dataset of stroke-related CT and MRI medical imaging data using a deep learning architecture, for detecting and classifying stroke-related characteristics to generate an analysis result; and a result generation module for producing a diagnostic result from the analysis result, displayed on the user interface. The stroke AI-assisted interpretation system, capable of processing multimodal data, analyzes a patient's medical imaging data to assist users in making faster, more accurate, and objective stroke diagnoses in a clinical environment.

### BACKGROUND OF THE INVENTION

### Technical Field

The present invention relates generally to a device that applies artificial intelligence to interpret data, and more particularly to an AI-assisted stroke interpretation system for evaluating and diagnosing medical imaging.

### Description of Related Art

Stroke is one of the leading causes of death and disability worldwide, and the timeliness of its diagnosis and treatment is crucial for improving patient outcomes. Medical imaging techniques, such as computed tomography (CT) and magnetic resonance imaging (MRI), produce medical images that are primary tools for diagnosing stroke in clinical practice. However, the interpretation of these medical images heavily relies on the clinical physician's expertise and may be affected by individual knowledge differences and fatigue, leading to inconsistencies in diagnostic results. Furthermore, delays in the diagnostic process when dealing with acute stroke patients may cause patients to miss the golden treatment time, thus affecting the treatment effect.

Although some auxiliary diagnostic software based on medical image analysis have been put into use, these tools are usually aimed at a single imaging modality (such as analyzing only CT or MRI medical images) and there are still limitations in detection accuracy, result interpretability, and clinical integration. For example, existing systems may not effectively combine multimodal data from CT and MRI medical images for comprehensive analysis. Additionally, most tools lack the ability to express model uncertainty, which is crucial for clinicians in assessing the reliability of results. Therefore, there is an urgent need for an artificial intelligence (AI) interpretation system that can overcome the aforementioned challenges, seamlessly integrate into clinical environments, and assist users in making faster, more accurate, and objective stroke diagnoses.

### BRIEF SUMMARY OF THE INVENTION

In view of the above, the primary objective of the present invention is to provide an advanced artificial intelligence (AI) interpretation system capable of analyzing multimodal data (CT and MRI medical images), automatically detecting the type of stroke in the medical imaging data, and assisting users in making faster, more accurate, and objective stroke diagnoses in clinical environments.

The present invention provides an AI-assisted stroke interpretation system, configured to operate on a terminal device including a user interface; the AI-assisted stroke interpretation system comprising an image acquisition module, an image preprocessing module, an artificial intelligence engine, and a result generation module. An AI-assisted stroke interpretation system is configured to operate on a terminal device including a user interface; wherein the image acquisition module receiving a medical imaging data from an imaging scanning equipment scanning a patient's computed tomography (CT) and/or magnetic resonance imaging (MRI); wherein the image preprocessing module is connected to the image acquisition module, and preprocessing the medical imaging data; wherein the artificial intelligence engine is trained on a deep learning architecture using a stroke-related medical imaging dataset, the artificial intelligence engine is used to detect and classify stroke-related characteristics; the stroke-related medical imaging dataset includes medical images of computed tomography and magnetic resonance imaging; the artificial intelligence engine is connected to the image preprocessing module for detecting and classifying stroke-related characteristics in the preprocessed medical imaging data, generating an analysis result; and wherein the result generation module is connected to the artificial intelligence engine, generating a diagnostic result from the analysis result, the diagnostic result displayed on the user interface.

According to the above aspect, the result generation module further includes a confidence scoring mechanism for indicating the certainty of the diagnostic result on the user interface.

According to the above aspect, the diagnostic result is categorized as acute, subacute, or chronic stroke based on the imaging features of the medical imaging data and temporal changes.

According to the above aspect, the artificial intelligence engine supports an active learning and feedback mechanism; the artificial intelligence engine detects and classifies stroke-related characteristics extracted from the medical imaging data, including quantitative indicators of lesion location, lesion size, and severity of the condition.

According to the above aspect, the analysis result is categorized as ischemic stroke or hemorrhagic stroke; the ischemic stroke includes the delineation of the ischemic core and penumbra; the hemorrhagic stroke includes morphology, location, and extent of the hemorrhagic stroke.

According to the above aspect, the result generation module converts the diagnostic result into a diagnostic report, the diagnostic report includes a stroke type, a spatial distribution, a severity, and a score of uncertainty regarding the results.

According to the above aspect, the diagnostic result displayed on the user interface, the diagnostic result includes an image overlay view, and the image overlay view is an image of a lesion area with stroke-related characteristics superimposed on a medical image of the medical imaging data.

According to the above aspect, the user interface is a touch display, allowing a user to add a real-time annotation to the image overlay view, the real-time annotation including a type of stroke, a size of the lesion, or a affected brain region.

According to the above aspect, the AI-assisted stroke interpretation system further comprising a feedback module, the feedback module being connected to the user interface and the artificial intelligence engine respectively, the feedback module is used to add the image overlay view including the real-time annotation to the stroke-related medical imaging dataset.

According to the above aspect, the image preprocessing module performs image preprocessing on the medical imaging data including noise elimination, intensity normalization, and anatomical structure segmentation.

With the abovementioned design, the effect of the present invention is that the stroke AI-assisted interpretation system relies on the detection and classification of the artificial intelligence engine that supports multi-modal medical imaging data. It could process CT medical images and MRI medical images and perform cross-modal comparisons. It has a more comprehensive diagnosis basis and can interpret the stroke-related characteristics of the patient's medical imaging data, and then generates the diagnosis result and displays the diagnosis result on the user interface. In this way, the diagnostic results are consistent and not affected by personal knowledge differences and fatigue, and the diagnosis will not be delayed to cause acute stroke patients to miss the golden time. In addition, the generated diagnostic report is displayed on the user interface and can be used as an auxiliary diagnostic tool for users, such as clinicians, to review and evaluate the reliability of the diagnostic results.

A further effect of the present invention is to classify the analysis result into ischemic stroke or hemorrhagic stroke; to distinguish the diagnosis result into acute, subacute and chronic stroke based on the imaging features of the medical imaging data and temporal changes; or the diagnostic report includes the diagnosis report including stroke type, spatial distribution, severity, and score of uncertainty regarding the results. In this way, the deep learning architecture of the artificial intelligence engine can be used to automatically detect and classify stroke types, quantify stroke severity and the spatial distribution of lesions in the brain, provide visualization and uncertainty assessment of diagnostic results, and help clinicians make more informed diagnostic decisions.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a block diagram of an AI-assisted stroke interpretation system according to a preferred embodiment of the present invention; and

FIG. 2 is a schematic view of a user interface of the preferred embodiment of the AI-assisted stroke interpretation system of the present invention.

### DETAILED DESCRIPTION OF THE INVENTION

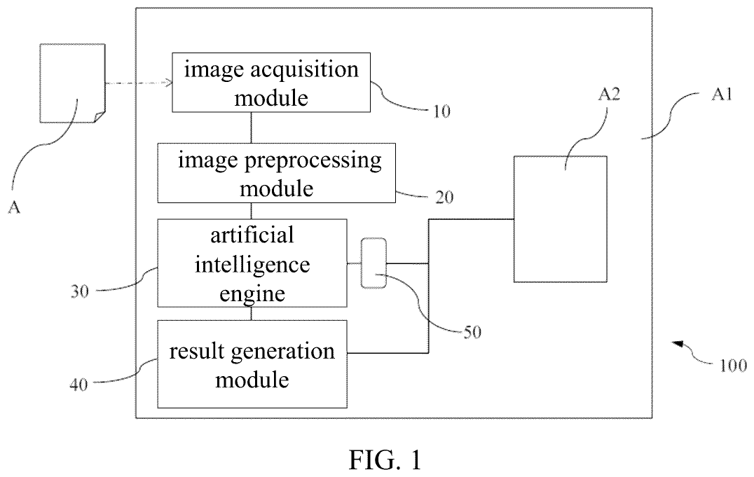

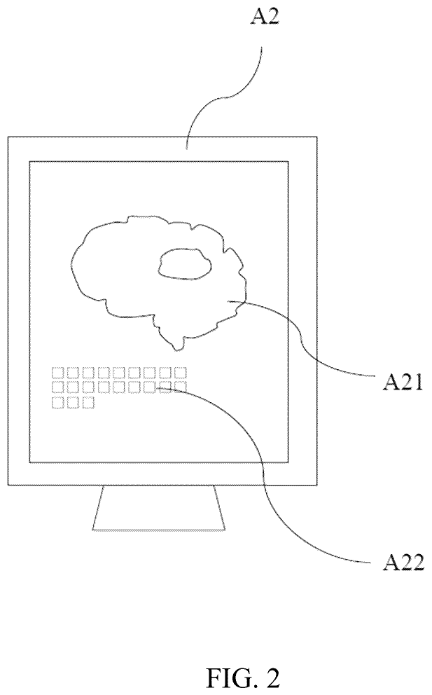

Please refer to FIG. 1 and FIG. 2, which illustrate a preferred embodiment of the AI-assisted stroke interpretation system 100 of the present invention. The AI-assisted stroke interpretation system 100 is configured to operate on a terminal device A1. Specifically, the AI-assisted stroke interpretation system 100 is implemented by storing instructions in a computer-readable medium of the terminal device A1, such as a hard disk, a tape, a solid-state drive, a flash memory, or a random-access memory.

In the preferred embodiment, the terminal device A1 is a computer or server, and the terminal device A1 includes a user interface A2 with input and output functionalities, allowing users, such as clinical physicians, to operate it. Specifically, the user interface A2 is a touch display. In other preferred embodiments, the terminal device A1 may be a tablet, smartphone, a self-service kiosk, or an industrial computer. In addition to being the touch display, the user interface A2 can also be a combination of a display, a keyboard, and a mouse

The stroke AI-assisted diagnosis system 100 further includes:

An image acquisition module 10, wherein the image acquisition module 10 receives a medical image data A from computerized tomography (CT) and/or magnetic resonance imaging (MRI) of a patient scanned by an imaging scanning device, and the image acquisition module 10 integrates multi-modal medical image data A for subsequent processing. The image acquisition module 10 is compatible with input formats of different image scanning devices and transmission data protocols, ensuring that the image acquisition module 10 can successfully receive the medical image data A.

An image preprocessing module 20, wherein the image preprocessing module 20 is coupled to the image acquisition module 10, and the image preprocessing module 20 performs image preprocessing on the medical imaging data A. The image preprocessing module 20 is designed to accommodate preprocessing techniques for multimodal data and is capable of intensity normalization, ensuring the uniformity of the medical imaging data A from computed tomography and magnetic resonance imaging for subsequent detection and classification, allowing for cross-modal comparisons. When the image preprocessing module 20 processes the medical imaging data A, it performs image processing that includes noise reduction, intensity normalization, anatomical structure segmentation, and artifact removal, with anatomical structure segmentation enhancing the visibility of lesions. The aforementioned image preprocessing aims to improve image quality, extract and enhance features relevant to stroke diagnosis, and ensure the consistency of the quality of the medical imaging data A.

An artificial intelligence engine 30, based on a deep learning architecture such as convolutional neural networks or transformers, the artificial intelligence engine 30 is trained using a dataset of stroke-related medical imaging dataset to detect and classify stroke-related characteristics. The stroke-related medical image dataset includes computed tomography (CT) scans and magnetic resonance imaging (MRI) from multiple institutions to ensure robustness under different scanners and protocols. The medical image dataset includes medical images of cases with expert-annotated ischemic core and penumbra regions. When used to detect and analyze the medical image data A, if a hemorrhagic stroke is determined, the ischemic core and penumbra are regionally located. The artificial intelligence engine 30 is configured to include an AI model for assessing ischemia and an AI model for evaluating hemorrhagic stroke indicators.

The artificial intelligence engine 30 is coupled with the image preprocessing module 20, allowing the medical image data A preprocessed from the image preprocessing module 20 to be input into the artificial intelligence engine 30. The artificial intelligence engine 30 detects and classifies stroke-related characteristics in the medical image data A preprocessed from the image preprocessing module 20, the stroke-related characteristics includes indicators of ischemic stroke and hemorrhagic stroke. The artificial intelligence engine 30 also classifies strokes into acute, subacute, and chronic stages, and quantifies the detected biomarkers to assess the severity of the stroke, resulting in an analysis result.

The analysis result includes the presence, type, location, and stage of the stroke. The type of stroke is categorized as ischemic stroke or hemorrhagic stroke. The location of the ischemic stroke includes the localization of the ischemic core and penumbra; the location of the hemorrhagic stroke includes the morphology, location, and extent of the hemorrhagic stroke. The image preprocessing module 20 detects and classifies the stroke-related characteristics extracted from the medical image data A, including lesion location, lesion size, and quantifiable indicators of severity. The image preprocessing module 20 supports active learning and feedback mechanisms, allowing users, such as clinical physicians, to add annotated cases to the medical image dataset for the continuous optimization and performance enhancement of the AI model, adapting to new medical image data and clinical needs.

A result generation module 40 is coupled with the artificial intelligence engine 30 and receives the analysis result from the artificial intelligence engine 30. The result generation module 40 generates a diagnostic result based on the analysis results, the diagnostic result is categorized as acute, subacute, and chronic strokes according to the imaging features and temporal changes of the medical image data. The result generation module 40 is coupled with the user interface A2, the result generation module 40 displays the diagnostic result along with a visualization of the stroke-related brain regions on the user interface A2 for users, such as clinical physicians, to use in clinical interpretation. The result generation module 40 further includes a confidence scoring mechanism to indicate the certainty of the diagnostic result for users, such as clinical physicians, on the user interface A2.

The user interface A2 can display quantitative data of lesions in real-time and receive user edits to the quantitative data. When the diagnostic result is displayed on the user interface A2, the diagnostic result includes an image overlay view A21, which overlays images of lesion areas with stroke-related features on the medical image data A. The user interface A2 allows users to add a real-time annotation A22 to the image overlay view A21, the real-time annotation A22 includes the type of stroke, the size of the lesion, or the affected brain regions, for real-time annotation of the type of stroke, lesion size, or affected brain regions during clinical interpretation by users, such as clinical physicians.

The result generation module 40 converts the diagnostic result into a diagnostic report, formatted as structured data that is compatible with the hospital's Picture Archiving and Communication System (PACS) for seamless integration, facilitating clinical integration and subsequent research. The diagnostic report includes types of stroke (ischemic or hemorrhagic), spatial distribution (affected brain regions), severity (volumes of the ischemic core and penumbra), and uncertainty scoring of the results (e.g., diagnostic confidence). The spatial distribution is visualized alongside the uncertainty scoring of the results, supporting clinical physicians in quickly understanding and making decisions. Since the diagnostic report is derived from the detection and analysis performed by the artificial intelligence engine 30 based on a deep learning architecture, it enhances the accuracy of stroke detection and classification judgments in the diagnostic report.

A feedback module 50 is coupled with both the user interface A2 and the artificial intelligence engine 30, the feedback module 50 is used to add the image overlay view A21 containing the real-time annotation A22 to the stroke-related medical image dataset. The user interface A2 supports interactive features, allowing clinical physicians to adjust the analysis results and provide new annotated data back to the system for model updates. This enables the artificial intelligence engine 30 to improve the performance of the AI model through the aforementioned feedback loop by actively learning from images of new cases.

As described above, the stroke AI-assisted interpretation system 100 architecture includes the terminal device A1 with the user interface A2, as well as the image acquisition module 10, the image preprocessing module 20, the artificial intelligence engine 30, the result generation module 40, and the feedback module 50. The stroke AI-assisted interpretation system 100 is used to assist in interpreting the patient's medical image data A. After acquiring the patient's computed tomography (CT) and magnetic resonance imaging (MRI) medical image data A through the image preprocessing module 20, the images undergo preprocessing steps via the image preprocessing module 20 to enhance features related to stroke diagnosis. The medical image data A after preprocessed is then input into the trained artificial intelligence engine 30 for detection, analysis, and generates the analysis results. The result generation module 40 then produces the diagnostic result based on the analysis result, which are ultimately presented to clinical physicians for interpretation through the user interface A2, or displayed to readers in a visualized and structured format via the generated diagnostic report. This enables the stroke AI-assisted interpretation system 100 to serve as an effective diagnostic tool, assisting clinical physicians in quickly understanding the patient's stroke condition and making more comprehensive diagnostic decisions. The diagnostic results can also facilitate clinical integration for subsequent case studies.

It must be pointed out that the embodiment described above is only a preferred embodiment of the present invention. All equivalent structures which employ the concepts disclosed in this specification and the appended claims should fall within the scope of the present invention.

What is claimed is:

1. An AI-assisted stroke interpretation system, configured to operate on a terminal device including a user interface; the AI-assisted stroke interpretation system comprising:
an image acquisition module, wherein the image acquisition module receiving a medical imaging data from an imaging scanning equipment scanning a patient's computed tomography (CT) and/or magnetic resonance imaging (MRI);
an image preprocessing module, wherein the image preprocessing module is connected to the image acquisition module, and preprocessing the medical imaging data;
an artificial intelligence engine, wherein the artificial intelligence engine is trained on a deep learning architecture using a stroke-related medical imaging dataset, the artificial intelligence engine is used to detect and classify stroke-related characteristics; the stroke-related medical imaging dataset includes medical images of computed tomography and magnetic resonance imaging; the artificial intelligence engine is connected to the image preprocessing module for detecting and classifying stroke-related characteristics in the preprocessed medical imaging data, generating an analysis result; and
a result generation module, wherein the result generation module is connected to the artificial intelligence engine, generating a diagnostic result from the analysis result, the diagnostic result displayed on the user interface.

2. The AI-assisted stroke interpretation system as claimed in claim 1, wherein the result generation module further includes a confidence scoring mechanism for indicating the certainty of the diagnostic result on the user interface.

3. The AI-assisted stroke interpretation system as claimed in claim 1, wherein the diagnostic result is categorized as acute, subacute, or chronic stroke based on the imaging features of the medical imaging data and temporal changes.

4. The AI-assisted stroke interpretation system as claimed in claim 1, wherein the artificial intelligence engine supports an active learning and feedback mechanism; the artificial intelligence engine detects and classifies stroke-related characteristics extracted from the medical imaging data, stroke-related characteristics including quantitative indicators of lesion location, lesion size, and severity of the condition.

5. The AI-assisted stroke interpretation system as claimed in claim 1, wherein the analysis result is categorized as ischemic stroke or hemorrhagic stroke; the ischemic stroke includes regional localization of the ischemic core and penumbra; the hemorrhagic stroke includes morphology, location, and extent of the hemorrhagic stroke.

6. The AI-assisted stroke interpretation system as claimed in claim 1, wherein the result generation module converts the diagnostic result into a diagnostic report, the diagnostic report includes stroke type, spatial distribution, severity, and score of uncertainty regarding the results.

7. The AI-assisted stroke interpretation system as claimed in claim 1, wherein the diagnostic result displayed on the user interface, the diagnostic result includes an image overlay view, and the image overlay view is an image of a lesion area with stroke-related characteristics superimposed on a medical image of the medical imaging data.

8. The AI-assisted stroke interpretation system as claimed in claim 7, wherein the user interface is a touch display, allowing a user to add a real-time annotation to the image overlay view, the real-time annotation including a type of stroke, a size of the lesion, or a affected brain region.

9. The AI-assisted stroke interpretation system as claimed in claim 8, further comprising a feedback module, the feedback module being connected to the user interface and the artificial intelligence engine respectively, the feedback module is used to add the image overlay view including the real-time annotation to the stroke-related medical imaging dataset.

10. The AI-assisted stroke interpretation system as claimed in claim 1, wherein the image preprocessing module performs image preprocessing on the medical imaging data including noise elimination, intensity normalization, and anatomical structure segmentation.
